(19) United States
(12) Patent Application Publication
(10) Pub. No.: US 2023/0234026 A1
(43) Pub. Date: Jul. 27, 2023

(54) CELLULOSE POROUS GEL MICROSPHERE WITH UNIFORM PARTICLE SIZE, PREPARATION METHOD AND APPLICATION

(71) Applicant: Zhejiang NUPTEC Biomacromolecular Purification Resins Manufacturing Inc. Ltd., Hangzhou (CN)

(72) Inventors: Zefeng Shi, Hangzhou (CN); Xianpeng Yang, Hangzhou (CN); Yongchang Qian, Hangzhou (CN)

(21) Appl. No.: 18/127,355

(22) Filed: Mar. 28, 2023

(30) Foreign Application Priority Data
Dec. 31, 2022 (CN) 202211739528.1

(51) Int. Cl. B01J 20/24; B01J 20/28; B01J 20/291; B01J 20/30; C07K 1/16; B01D 15/08

(52) U.S. Cl. CPC B01J 20/24; B01J 20/28047; B01J 20/28019; B01J 20/291; B01J 20/3028; B01J 20/3085; C07K 1/16; B01D 15/08; B01J 2220/52

(57) ABSTRACT

The present invention provides a cellulose porous gel microsphere with uniform particle size, a preparation method and application. Based on the liquid-liquid dispersion theory and the innovation of the underlying technology, the present invention proposes the preparation of high-performance cellulose porous gel microspheres by cellulose acetate solution with low viscosity. The present invention is environmental-friendly, low in requirements for equipment, low in cost, and easy for expanded production and application. The cellulose acetate with low viscosity is used as the raw material, and the prepared cellulose porous gel microspheres have high sphericity, uniform particle size, moderate microsphere pore size, high mechanical strength and excellent pressure/flow rate performance and are suitable for the modification of various ligands and the separation and the purification of various biomacromolecules in various modes after modification. The present invention can compete with agarose porous gel microspheres and can realize efficient separation of the biomacromolecules in chromatography.

### TECHNICAL FIELD

The present invention belongs to the technical field of microsphere preparation, relates to a chromatographic medium, and particularly relates to a cellulose porous gel microsphere with uniform particle size, a preparation method and application.

### BACKGROUND

The chromatographic technique has become the most commonly used separation and purification means in the production process of large-scale biological products because of its high separation accuracy, mild separation conditions, simple operation and high repeatability. A chromatographic medium is the core basic material of bioseparation. Porous gel microspheres based on natural polymers such as agarose and cellulose are the most widely used chromatographic media for bioseparation in industry because of the characteristics of few soluble substances, high biosecurity, easy modification of functional ligands and weak non-specific adsorption. The advantages of agarose for preparation of the porous gel microspheres are good water solubility, appropriate gelation temperature, large gel strength and easy regulation of pore structure. At present, agarose porous gel microspheres dominate the market.

Compared with the agarose, the price of cellulose raw materials is low, and cellulose chains can form crystalline structure with higher frame strength. Since the 1990s, new solvents such as N-methylmorpholine oxide, ionic liquid and alkali/urea solution have appeared, and the dissolution problem of cellulose has been solved to some extent. In recent years, the development speed of cellulose porous gel microspheres is higher than that of agarose porous gel microspheres. WO1999031141A2, WO2012033223A1 and WO2017141910A1 patents disclose the preparation methods of porous gel microspheres based on the above novel cellulose solvent, to avoid the early use of thiocyanate, carbon disulfide and other toxic solvents. The cellulose porous gel microspheres have been developed greatly, but there are still challenges such as high system viscosity, expensive solvent, high equipment requirements and poor sphericity of the obtained microspheres.

Compared with cellulose, cellulose derivatives can also be used as raw materials of the cellulose porous gel microspheres due to wider selection of alternative solvents. WO2016013568A1 and WO2015029790A1 patents disclose the methods for preparing cellulose porous gel microspheres using cellulose acetate as raw material. However, the obtained microspheres are difficult to simultaneously satisfy the new requirements of bioseparation such as uniform particle size, high rigidity, high flow rate and high load.

### SUMMARY

A first purpose of the present invention is to provide a preparation method of cellulose porous gel microsphere with uniform particle with respect to the above defects.

Thus, the above purpose of the present invention is achieved through the following technical solution:

A preparation method of cellulose porous gel microspheres with uniform particle size, comprising:

S1. dissolving cellulose acetate in an organic solvent-water mixed solvent as an aqueous phase:

dissolving 7-14 parts of cellulose acetate in 100 parts of organic solvent-water mixed solvent to form a cellulose acetate solution with moderate viscosity at dissolving temperature of 60-90° C., so that the cellulose acetate is dissolved at the dissolving temperature and semi-solid substances can be formed after cooling; the organic solvent is a good solvent of cellulose acetate and is miscible with water;

S2. preparing cellulose acetate gel microspheres by a phase inversion emulsification method:

pouring the aqueous phase for dissolving the cellulose acetate into an oil phase heated to 60-90° C.; emulsifying the mixture by mechanical stirring (preferably, emulsifying by stirring for 10-30 minutes), so that the aqueous phase is dispersed into droplets with required particle size; cooling (preferably, cooling to below 20° C.) the emulsion so that the aqueous phase droplets form semi-solid substances; and after cleaning, obtaining cellulose acetate gel microspheres;

the oil phase contains an inert solvent and an emulsifier, and the inert solvent is not miscible with the organic solvent and the water in step S1;

the emulsifier is a non-ionic surfactant; the emulsifier is a single emulsifier or a compound emulsifier with an HLB value of 3-8; the ratio of the emulsifier in the oil phase to the inert solvent is 0.1-5 w/v %; and the volume ratio of the aqueous phase to the oil phase is 1:(2-5);

S3. saponifying and crosslinking the cellulose acetate gel microspheres:

saponifying the cellulose acetate gel microspheres into cellulose gel microspheres under alkaline conditions, and crosslinking the cellulose gel microspheres with epichlorohydrin, wherein the volume dosage of the epichlorohydrin is 1-20% of the volume of the microspheres, to obtain the crosslinked cellulose porous gel microspheres.

While the above technical solution is adopted, the present invention may also adopt or combine the following technical solutions:

As a preferred technical solution of the present invention: the degree of substitution of the cellulose acetate is preferably 0.5-2.5; and after cellulose acetate forms a solution, the degree of substitution and the molecular weight of the cellulose acetate can be adjusted by adding alkaline substances such as hexamethylene diamine.

As a preferred technical solution of the present invention: the organic solvent in the organic solvent-water mixed solvent is at least one of acetone, dimethyl sulfoxide and N,N-dimethylformamide.

As a preferred technical solution of the present invention: the inert solvent is at least one of cyclohexane or liquid paraffin.

As a preferred technical solution of the present invention: the emulsifier is at least one of Span 85, Span 80 and Span 60.

As a preferred technical solution of the present invention: the HLB of the emulsifier is regulated by Tween 80.

As a preferred technical solution of the present invention: the ratio of the emulsifier in the oil phase to the inert solvent is preferably 0.1-1 w/v %.

As a preferred technical solution of the present invention: the volume dosage of the epichlorohydrin is preferably 5-15% of the volume of cellulose porous gel.

As a preferred technical solution of the present invention: in step S3, NaOH is added to achieve alkaline conditions.

A second purpose of the present invention is to provide a cellulose porous gel microsphere with uniform particle size, which is prepared by the above preparation method of cellulose porous gel microspheres with uniform particle size.

While the above technical solution is adopted, the present invention may also adopt or combine the following technical solutions:

As a preferred technical solution of the present invention: there is a crystalline structure in the cellulose acetate, and the crystalline structure is cellulose type II.

A third purpose of the present invention is to provide a chromatographic medium of cellulose porous gel microspheres with uniform particle size, which is obtained by modifying the above cellulose porous gel microspheres with uniform particle size by using ligand.

Another purpose of the present invention is to provide application of the above cellulose porous gel microspheres with uniform particle size in separation and purification aspects of biomacromolecules. After modified by the ligand, the above cellulose porous gel microspheres with uniform particle size is used as a chromatographic medium for separation and purification of biomacromolecules such as protein and nucleic acid.

The separation and purification modes of the biomacromolecules are not especially restricted, and may be common chromatography modes such as affiliation, hydrophobic interaction, ion exchange, gel filtration and mixing.

The present invention provides a cellulose porous gel microsphere with uniform particle size, a preparation method and application. Based on the liquid-liquid dispersion theory and the innovation of the underlying technology, the present invention proposes the preparation of high-performance cellulose porous gel microspheres by cellulose acetate solution with low viscosity. The cellulose porous gel microspheres obtained by the present invention are close to a regular spheroid, the particle size is 45-165 μm, and the Span value of a particle size distribution coefficient is less than 0.90; the cellulose porous gel microspheres are porous with porosity greater than 90%; dextran with the molecular weight of 102 K as a marker has a gel distribution coefficient of 0.35-0.55 and solid content of 5-12%; and the cellulose porous gel microspheres have withstand voltage of greater than 0.2 MPa, and maximum stable flow rate of greater than 1500 cm/h. The present invention is environmental-friendly, low in requirements for equipment, low in cost, and easy for expanded production and application. The cellulose acetate with low viscosity is used as the raw material, and the prepared cellulose porous gel microspheres have high sphericity, uniform particle size, moderate microsphere pore size, high mechanical strength and excellent pressure/flow rate performance and are suitable for the modification of various ligands and the separation and the purification of various biomacromolecules in various modes after modification. The present invention can compete with agarose porous gel microspheres and can realize efficient separation of the biomacromolecules in chromatography.

### DESCRIPTION OF DRAWINGS

FIG. 1 is a microphotograph of cellulose porous gel microsphere with uniform particle size provided by the present invention.

FIG. 2 is a particle size distribution chart of cellulose porous gel microsphere with uniform particle size provided by the present invention.

FIG. 3 shows pressure/flow rate characteristic curves of cellulose porous gel microsphere with uniform particle size provided by the present invention.

### DETAILED DESCRIPTION

The present invention will be further described below in detail in combination with specific embodiments, but the implementation modes of the present invention are not limited thereto.

The key raw material of the present invention is cellulose acetate, and the viscosity of cellulose acetate solution is an important influence factor that controls the particle size uniformity of the obtained cellulose porous gel microspheres. A simple way to control the viscosity of the cellulose acetate solution is to control the dosage and the molecular weight of cellulose acetate, and the molecular weight is adjusted by adding alkaline substances (e.g., hexamethylene diamine).

In the following embodiments, cellulose acetate is purchased from Shanghai Aladdin Bio-Chem Technology Co., Ltd., with item No. C106244 and degree of substitution of 2.5. The degree of substitution and the molecular weight are regulated by adding hexamethylene diamine.

### Embodiment 1

A method of cellulose porous gel microspheres with uniform particle size comprises the following steps:

S1. dissolving cellulose acetate in an organic solvent-water mixed solvent as an aqueous phase:

weighing 10.0 g of cellulose acetate and dissolving in 30.0 g of dimethyl sulfoxide; heating to 80° C. while stirring; dropwise adding 14.0 g of hexamethylene diamine aqueous solution with concentration of 30% into the solution; stirring for 120 min, and then adding 54.0 g of water; stirring for 30 min for later use; and reducing the degree of substitution and the molecular weight of the cellulose acetate so that the aqueous phase is suitable for phase inversion emulsification;

S2. preparing cellulose acetate gel microspheres by a phase inversion emulsification method:

adding 0.2 g of Span 80 emulsifier and 200 ml of liquid paraffin to a 500 mL three-necked round-bottomed flask, and heating to 80° C. as an oil phase for later use; adding the aqueous phase in step 1 to the oil phase for emulsification at emulsification speed of 500 rpm, emulsification temperature of 80° C. and emulsification time of 20 min; after emulsification, cooling the emulsion to below 20° C. at a rate of 2° C./min to form gel microspheres; and washing to obtain 50 mL of gel microspheres;

S3. saponifying and crosslinking the cellulose acetate gel microspheres:

adding ethanol to the microspheres in step S2, and making the volume ratio of the ethanol as 40%; dropwise adding 200 ml of ethanol aqueous solution containing 0.4% of NaOH; saponifying overnight at 15° C.; and washing to obtain cellulose microspheres; taking 50.0 g of cellulose microspheres, adding 37.5 mL of Na₂SO₄ with concentration of 2.5 M, 1.0 mL of NaOH with mass fraction of 45% and 0.075 g of NaBH₄ successively; after stirring at 50° C. for 1 h, adding 0.6 mL of NaOH with mass fraction of 45% and 0.625 mL of epichlorohydrin every 15 min for a total of 8 times of adding dropwise; after adding dropwise, heating to 60° C.; continuing the reaction for 16 h; after the reaction is finished, washing with deionized water to obtain crosslinked cellulose porous gel microspheres.

### Particle Size Test of Cellulose Porous Gel Microspheres

The particle size and the distribution of the crosslinked cellulose porous gel microspheres are tested by Omec LS-POP(9) laser particle size analyzer. The obtained parameters are D10, D50, D90 and the Span value of the particle size distribution coefficient, wherein the distribution of D10, D50 and D90 indicates that the volume of all microspheres with smaller diameters accounts for 10%, 50% and 90% of the total volume of all the microspheres, and the particle size distribution coefficient is Span=D₉₀−D₁₀/D₅₀. D50 represents the average particle size of the microspheres, and the Span value represents the uniformity of particle size distribution. In the present invention, the Span value greater than 1.50 is defined as wide particle size distribution and poor uniformity of particle size distribution; the Span value between 1.20 and 1.50 is defined as wide particle size distribution; the Span value between 0.90 and 1.20 is defined as narrow particle size distribution; and the Span value less than 0.90 is defined as narrow particle size distribution and good uniformity of particle size distribution.

### Solid Content Test of Cellulose Porous Gel Microspheres

About 10 g of wet spheres are placed in gravity column and drained. About 3 g of wet spheres are accurately weighed and dried in an oven at 105° C. to reach constant weight. The ratio of the mass of the dried spheres to the mass of the wet spheres is solid content.

### Pressure/Flow Rate Characteristic Test of Cellulose Porous Gel Microspheres

Instrument: protein purifier of SCG-100 protein chromatography system

Chromatographic column: cytiva Tricorn 10/100 Column

Mobile phase: pure water

Test: 8 mL of crosslinked cellulose porous gel microspheres are loaded into the above chromatographic column, the flow rate starts from 0.5 mL/min, and the pressure is detected. The flow rate is gradually increased every 5 min until the system pressure is sharply increased to 3 MPa, indicating that the sample is collapsed and the flow rate cannot be increased further, thereby ending the test. The increasing sequence of the flow rate is 0.5, 1.0, 1.5, 2.0, 3.5, 4.0, 4.5, 5.0, 6.0, 7.0, 8.0, 9.0, 10.0, 12.0, 14.0, 16.0, 18.0 . . . 66.0 mL/min successively. The volume flow rate is converted into linear velocity: V=(60×V_v)/S; V is the linear velocity (cm/h), V_v is the volume flow rate (mL/min), and S is the cross-sectional area 0.785 cm² of the chromatographic column. The flow rate and the column pressure at the last section of stable pressure before sharp rise in pressure are defined as the maximum flow rate and withstand pressure of the porous gel microspheres.

### Test of Gel Distribution Coefficient Kₐᵥ of Cellulose Porous Gel Microspheres

Instrument: protein purifier of SCG-100 protein chromatography system

Chromatographic column: Cytiva Tricorn 10/200 Column

Mobile phase: pure water, 0.8 mL/min

Sampling: after the crosslinked cellulose porous gel microspheres are loaded into the column, 20 μL of blue glucan with concentration of 3 mg/mL, 10 mg/mL glucan standard with molecular weight of 102 K, 8 mg/mL glucan standard with molecular weight of 64 K, 8 mg/mL glucan standard with molecular weight of 13 K and 2% acetone are sampled successively. For glucan with different molecular weights, Kₐᵥ=(Vₑ−V₀)/(Vₜ−V₀), where Vₑ is the retention volume (ml) of the glucan standards with different molecular weights, Vₜ is the retention volume (ml) of acetone and V₀ is the retention volume (ml) of blue glucan.

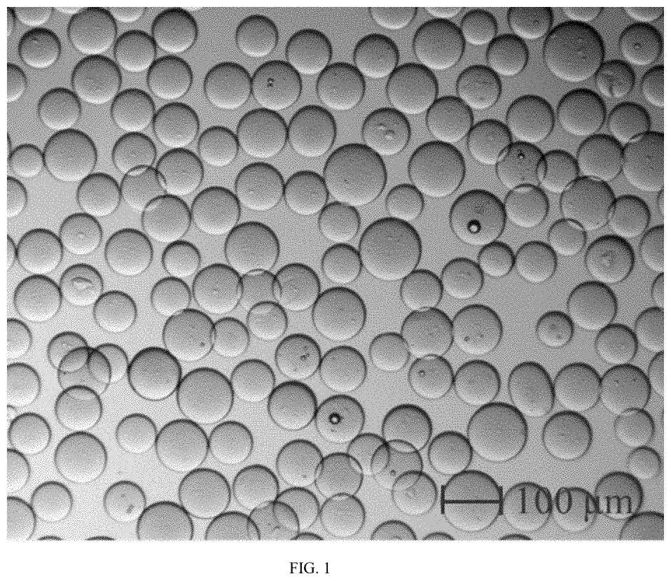

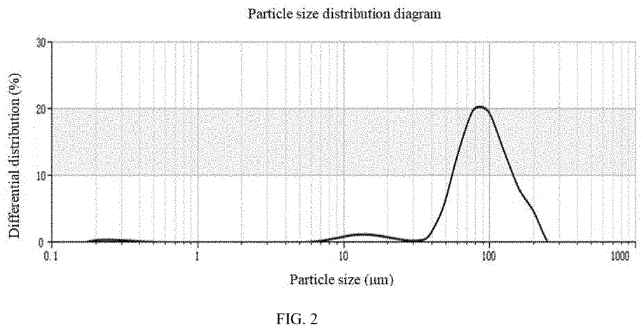

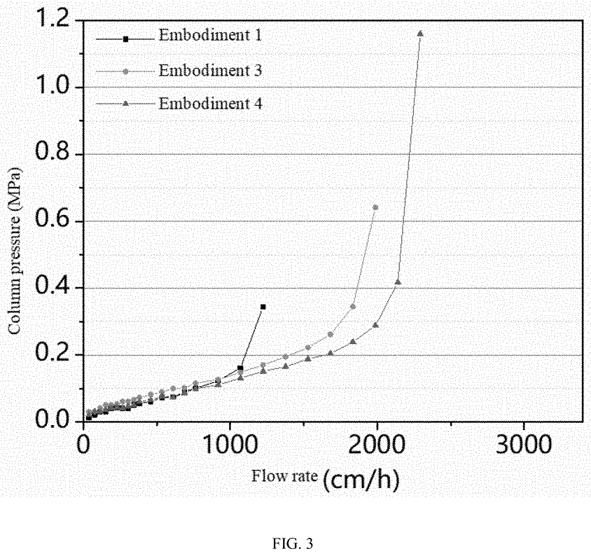

The unscreened D10, D50 and D90 of the crosslinked cellulose porous gel microspheres obtained in embodiment 1 are 38 μm, 72 μm and 112 μm, respectively, and the Span value is 1.03. The screened D10, D50 and D90 are 51 μm, 85 μm and 120 μm, respectively, and the Span value is 0.81. The microphotograph is shown in FIG. 1. Particle size distribution is shown in FIG. 2. The solid content of the microspheres is 8.0%. After screening, the maximum flow rate of the microspheres is 1070 cm/h, the withstand pressure is 0.16 MPa and the pressure/flow rate characteristic curves are shown in FIG. 3. The Kₐᵥ values of the glucan with molecular weights of 102 K, 64 K and 13 K are 0.45, 0.53 and 0.81 respectively.

### Embodiment 2

In preparation of the cellulose porous gel microspheres, step 1 in embodiment 1 is changed to the following conditions, the emulsification speed in step 2 is changed to 400 rpm, reagent dosages in other steps are changed in proportion, and experimental conditions such as temperature and reaction time are unchanged;

S1. dissolving cellulose acetate in an organic solvent-water mixed solvent as an aqueous phase:

weighing 4.0 g of cellulose acetate with acetyl content of 40% and dissolving in 15.0 g of dimethyl sulfoxide; heating to 80° C. while stirring; dropwise adding 6.0 g of hexamethylene diamine aqueous solution with concentration of 30% into the solution; stirring for 120 min, and then adding 29.0 g of water; stirring for 30 min for later use; and reducing the degree of substitution and the molecular weight of the cellulose acetate so that the aqueous phase is suitable for phase inversion emulsification.

The unscreened D10, D50 and D90 of the crosslinked cellulose porous gel microspheres obtained in embodiment 2 are 40 μm, 81 μm and 133 μm, respectively, and the Span value is 1.15. The crosslinked cellulose porous gel microspheres obtained in embodiment 2 have weak strength and are not suitable for pressure/flow rate test.

### Embodiment 3

In preparation of the cellulose porous gel microspheres, step 1 in embodiment 1 is changed to the following conditions, the emulsification speed in step 2 is changed to 440 rpm and other conditions are unchanged.

S1. dissolving cellulose acetate in an organic solvent-water mixed solvent as an aqueous phase:

weighing 11.0 g of cellulose acetate with acetyl content of 40% and dissolving in 30.0 g of dimethyl sulfoxide; heating to 80° C. while stirring; dropwise adding 14.0 g of hexamethylene diamine aqueous solution with concentration of 30% into the solution; stirring for 60 min, and then adding water; stirring for 30 min continuously; and reducing the degree of substitution and the molecular weight of the cellulose acetate so that the aqueous phase is suitable for phase inversion emulsification.

The unscreened D10, D50 and D90 of the crosslinked cellulose porous gel microspheres obtained in embodiment 3 are 62 μm, 102 μm and 155 μm, respectively, and the Span value is 0.92 without the need for screening. The solid content of the microspheres is 8.8%, the maximum flow rate of the microspheres is 1700 cm/h, the withstand pressure is 0.26 MPa and the pressure/flow rate curves are shown in FIG. 3.

### Embodiment 4

In preparation of the cellulose porous gel microspheres, step 1 in embodiment 1 is changed to the following conditions, the emulsification speed in step 2 is changed to 440 rpm and other conditions are unchanged.

S1. dissolving cellulose acetate in an organic solvent-water mixed solvent as an aqueous phase:

weighing 12.0 g of cellulose acetate with acetyl content of 40% and dissolving in 30.0 g of dimethyl sulfoxide; heating to 80° C. while stirring; dropwise adding 14.0 g of hexamethylene diamine aqueous solution with concentration of 30% into the solution; stirring for 60 min, and then adding water; stirring for 30 min continuously; and reducing the degree of substitution and the molecular weight of the cellulose acetate so that the aqueous phase is suitable for phase inversion emulsification.

The unscreened D10, D50 and D90 of the crosslinked cellulose porous gel microspheres obtained in embodiment 4 are 81 μm, 134 μm and 210 μm, respectively, and the Span value is 0.96. The screened D10, D50 and D90 are 68 μm, 106 μm and 152 μm, respectively, and the Span value is 0.79. The solid content of the microspheres is 9.0%. After screening, the maximum flow rate of the microspheres is 2000 cm/h, the withstand pressure is 0.29 MPa and the pressure/flow rate curves are shown in FIG. 3. The Kₐᵥ values of the glucan with molecular weights of 102 K, 64 K and 13 K are 0.47, 0.55 and 0.83 respectively.

### Embodiment 5

In preparation of the cellulose porous gel microspheres, the oil phase of step 2 in embodiment 3 is changed to a mixed solvent of 160 mL of liquid paraffin and 40 mL of cyclohexane, the emulsification speed is changed to 560 rpm and other conditions are unchanged.

The unscreened D10, D50 and D90 of the crosslinked cellulose porous gel microspheres obtained in embodiment 5 are 52 μm, 93 μm and 147 μm, respectively, and the Span value is 1.02.

### Embodiment 6

In application of the cellulose porous gel microspheres, the crosslinked cellulose microspheres obtained in embodiment 1 are modified with nickel-iminodiacetic acid (Ni-IDA) ligands as affinity chromatography media, comprising the following steps:

1) Allylation modification. Weighing 10 g of crosslinked cellulose porous gel microspheres drained in a gravity column; adding 3 mL of Na₂SO₄ solution with concentration of 2.5 mol/L, 2 mL of NaOH solution with concentration of 30 wt % and 25 mg of NaBH₄; and slowly adding 2.5 mL of allyl glycidyl ether at 45° C. while stirring to react for 16 h.

2) Bromine water activation. Adding 3.5 mL of purified water and 2.0 g of sodium acetate into the allyl-modified cellulose porous gel microspheres; dropwise adding new bromine water while stirring at room temperature for activation, until the yellow color does not fade within 1 min; and then adding 0.04 g of sodium formate to remove the remaining bromine water.

3) IDA modification and Ni loading. Adding 10 mL of sodium iminodiacetate solution (15 wt %, 50% NaOH solution is used for adjusting pH=11.5) into the brominated product and reacting at 50° C. for 18 h; after IDA modification, loading Ni into agarose/cellulose nanocomposite porous gel microspheres by using 50 mmol/L NiSO₄ solution to obtain chromatographic media with Ni-IDA ligand.

### Dynamic Binding Load Test of Ni-IDA Chromatographic Media

Instrument: AKTA explorer 100 protein purification system

Chromatographic column: cytiva Tricorn 5/100 Column

Packing: 2.0 mL of Ni-IDA chromatographic media are loaded into the above chromatographic column; 3 CV (column volume) is balanced with buffer solution A; 2 mg/mL protein A solution with His label is used for sampling (0.33 mL/min); sampling is stopped when 10% of breakthrough is reached; and 3 CV is balanced with the buffer solution A and eluted with buffer solution B. The dynamic binding load is calculated by the following formula: DBC_{10%}=(V_{10%}−V₀)C₀/V_{c}; V_{10%} is a sampling volume at 10% breakthrough, V₀ is the dead pipeline volume of a detection system (2.33 ml), and V_{c} is the volume of microspheres in the column (2.0 ml).

The buffer solution A is composed of 16.2 mmol/L sodium phosphate dibasic dodecahydrate, 3.8 mmol/L sodium dihydrogen phosphate dihydrate, and 20 mmol/L sodium chloride, with pH=7.4.

The buffer solution B is composed of 16.2 mmol/L sodium phosphate dibasic dodecahydrate, 3.8 mmol/L sodium dihydrogen phosphate dihydrate, 20 mmol/L sodium chloride and 500 mmol/L imidazole, with pH=7.6.

In embodiment 6, the dynamic binding load of protein A of the cellulose porous gel microspheres modified with Ni-IDA is 43.1 mg/mL.

### Embodiment 7

In application of the cellulose porous gel microspheres, the crosslinked cellulose microspheres obtained in embodiment 3 are modified with diethylaminoethyl chloride (DEAE) ligands as ion exchange chromatographic media, comprising the following steps:

weighing 10 g of crosslinked cellulose porous gel microspheres drained in a gravity column; adding 4.0 g of NaOH solution with concentration of 6 M; after shaking and activating for 1 h at 50° C. in a shaker, adding 6.0 ml of DEAE hydrochloric acid solution with concentration of 3 M; continuing to shake for 3 h at 50° C. in the shaker; and then washing with deionized water to be neutral, to obtain DEAE modified crosslinked cellulose microspheres.

### Test of Ion Exchange Capacity

1 ml of cellulose DEAE microspheres are taken into a gravity column with a diameter of 7 mm, washed with 5.0 ml of HCl standard solution with concentration of 0.1 mol/L, made to flow slowly and then washed with deionized water to be neutral. The cellulose DEAE microspheres are washed with 5.0 ml of NaOH standard solution with concentration of 0.1 mol/L, made to flow slowly and then washed with deionized water to be neutral. The column is immersed with 5.0 ml of HCl standard solution with concentration of 0.1 mol/L for 20 min; the solution is made to slowly flow out; the effluent is collected; the effluent is washed with about 15 ml of deionized water, collected, and merged with the previous effluent; and two drops of phenolphthalein indicator are dripped. NaOH standard solution with concentration of 0.1 mol/L is used for titration until the color of the solution is changed and does not fade within 15 s, that is, the end point of titration, to obtain the volume V_{NaOH} of consumed NaOH. The ion exchange capacity

$$= \frac{{V}_{HC 1} {M}_{HC 1} - {V}_{NaOH} {M}_{NaOH}}{{V}_{microsphere}} * 1000$$

(μmol/mL), where V_{HCl} is the volume (mL) of standard HCl solution, M_{HCl} is the molar concentration (mol/L) of standard HCl solution, V_{NaOH} is the volume (mL) of standard NaOH, M_{NaOH} is the molar concentration (mol/L) of standard NaOH and V_{microsphere} is the volume (mL) of cellulose DEAE microsphere.

### Dynamic Binding Load Test of DEAE Chromatographic Media

Instrument: AKTA explorer 100 protein purification system

Chromatographic column: cytiva Tricorn 5/50 Column

Packing: 1.0 mL of Ni-IDA chromatographic media are loaded into the above chromatographic column; 3 CV (column volume) is balanced with buffer solution A; 1 mg/mL bovine serum albumin solution is used for sampling (0.50 mL/min); sampling is stopped when 10% of breakthrough is reached; and 10 CV is balanced with the buffer solution A and eluted with buffer solution B in a linear gradient. 10 CV reaches 100% buffer solution B. The dynamic binding load is calculated by the following formula: DBC_{10%}=(V_{10%}−V₀)C₀/V_{c}; V_{10%} is a sampling volume at 10% breakthrough, V₀ is the dead pipeline volume of a detection system (2.33 ml), and V_{c} is the volume of microspheres in the column (1.0 ml).

The buffer solution A is composed of 50 mmol/L Tris-HCl (pH=8.5).

The buffer solution B is composed of 50 mmol/L Tris-HCl and 2 mol/L NaCl (pH=8.5).

In embodiment 7, after the cellulose porous gel microspheres are modified with DEAE, the ion exchange capacity is 181 μmol/L and the dynamic binding load of bovine serum albumin is 109.5 mg/mL.

The above specific implementation modes are used for explaining the present invention and are preferred embodiments of the present invention only, not for limiting the present invention. Any modification, equivalent replacement, improvement, etc. made to the present invention within the spirit of the present invention and the protection scope of claims shall fall into the protection scope of the present invention.

1. A preparation method of cellulose porous gel microspheres with uniform particle size, comprising:
S1. dissolving cellulose acetate in an organic solvent-water mixed solvent as an aqueous phase:
dissolving 7-14 parts of cellulose acetate in 100 parts of organic solvent-water mixed solvent to form a cellulose acetate solution with moderate viscosity at dissolving temperature of 60-90° C., so that the cellulose acetate is dissolved at the dissolving temperature and semi-solid substances can be formed after cooling;
S2. preparing cellulose acetate gel microspheres by a phase inversion emulsification method:
pouring the aqueous phase for dissolving the cellulose acetate into an oil phase heated to 60-90° C.; emulsifying the mixture by mechanical stirring, so that the aqueous phase is dispersed into droplets with required particle size; cooling the emulsion so that the aqueous phase droplets form semi-solid substances; and after cleaning, obtaining cellulose acetate gel microspheres;
the oil phase contains an inert solvent and an emulsifier, and the inert solvent is not miscible with the organic solvent and the water in step S1;
the emulsifier is a non-ionic surfactant; the emulsifier is a single emulsifier or a compound emulsifier with an HLB value of 3-8; the mass of the emulsifier in the oil phase accounts for 0.1%-5% of the volume of the oil phase, and the volume ratio of the aqueous phase to the oil phase is 1:(2-5);
S3. saponifying and crosslinking the cellulose acetate gel microspheres:
saponifying the cellulose acetate gel microspheres into cellulose gel microspheres under alkaline conditions, and crosslinking the cellulose gel microspheres with epichlorohydrin, wherein the volume dosage of the epichlorohydrin is 1-20% of the volume of the microspheres, to obtain the crosslinked cellulose porous gel microspheres.

2. The preparation method of cellulose porous gel microspheres with uniform particle size according to claim 1, wherein the degree of substitution of the cellulose acetate is preferably 0.5-2.5.

3. The preparation method of cellulose porous gel microspheres with uniform particle size according to claim 1, wherein the organic solvent in the organic solvent-water mixed solvent is at least one of acetone, dimethyl sulfoxide and N,N-dimethylformamide.

4. The preparation method of cellulose porous gel microspheres with uniform particle size according to claim 1, wherein the inert solvent is at least one of cyclohexane or liquid paraffin.

5. The preparation method of cellulose porous gel microspheres with uniform particle size according to claim 1, wherein the emulsifier is at least one of Span 85, Span 80 and Span 60.

6. The preparation method of cellulose porous gel microspheres with uniform particle size according to claim 1, wherein the HLB of the emulsifier is regulated by Tween 80.

7. The preparation method of cellulose porous gel microspheres with uniform particle size according to claim 1, wherein the volume dosage of the epichlorohydrin is preferably 5-15% of the volume of cellulose porous gel.

8. Cellulose porous gel microspheres with uniform particle size, which are prepared by the preparation method of cellulose porous gel microspheres with uniform particle size of claim 1.

9. A chromatographic medium of cellulose porous gel microspheres with uniform particle size, which is obtained by modifying the cellulose porous gel microspheres with uniform particle size of claim 8 by using ligand.

10. Application of the cellulose porous gel microspheres with uniform particle size of claim 8 in separation and purification aspects of biomacromolecules.
